(19) United States
(12) Patent Application Publication
(10) Pub. No.: US 2005/0187212 A1
(43) Pub. Date: Aug. 25, 2005

(54) Pharmaceutical composition for topical delivery of meloxicam

(75) Inventors: Toshimitsu Ohki, Osaka (JP); Noritaka Seko, Osaka (JP); Naohisa Kawamura, Saitama (JP); Ken-Ichi Noguchi, Saitama (JP); Junji Kawakami, Saitama (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Nippon Boehringer Ingelheim Co., Ltd., Ingelheim (DE)

(21) Appl. No.: 11/045,233

(22) Filed: Jan. 27, 2005

Related U.S. Application Data
(63) Continuation of application No. PCT/JP03/11782, filed on Sep. 17, 2002.

(51) Int. Cl. A61K 31/5415

(52) U.S. Cl. 514/226.5

(57) ABSTRACT

A pharmaceutical composition comprising: (a) a pharmaceutically effective amount of meloxicam, or a pharmaceutically acceptable salt thereof; (b) a skin permeation enhancing agent selected from the group consisting of: (i) an amine of formula I 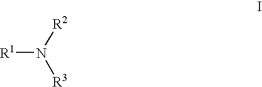
wherein: R¹, R², and R³ are each independently an alkanolyl group with 1 to 24 C-atoms, in which one or more H-atoms are optionally substituted by —OH and/or one or more —CH₂— groups are optionally substituted by —O—, or a polyoxyethylene group with 2 to 30 ethylene oxide units, and R² and R³ are additionally independently H or a linear, branched, or cyclic alkyl group with 1 to 24 C-atoms, in which one or more —CH₂— groups are optionally substituted by —CH═CH— and/or —O— and/or in which one or more H-atoms are optionally substituted by —OH, and (ii) an amide of formula II 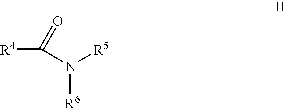
wherein: R⁴ and R⁵ are each independently a linear, branched, or cyclic alkyl group with 1 to 24 C-atoms, in which one or more —CH₂— groups are optionally substituted by —CH═CH—and/or —O— and/or in which one or more H-atoms are optionally substituted by —OH, and R⁶ is H or a linear, branched, or cyclic alkyl group with 1 to 24 C-atoms, in which one or more —CH₂— groups are optionally substituted by —CH═CH— and/or —O— and/or in which one or more H-atoms are optionally substituted by —OH; and (c) at least one inert carrier. Furthermore this invention relates to a transdermal delivery system and a method for treating, preventing and/or relieving the signs and/or symptoms of rheumatoid arthritis, cervico-omo-brachial syndrome, low back pain, osteoarthritis, periarthritis scapulohumeralis, tendovaginitis, peritendinitis, humerus epicondylitis, including tennis elbow, myalgia, post-traumatic tumor and pain.

### RELATED APPLICATIONS

This application is a continuation of International Application No. PCT/JP03/11782, filed on 17 Sep. 2002, benefit of which is hereby claimed, pursuant to 35 U.S.C. § 365(c) and § 120, and which is hereby incorporated by reference in its entirety.

### FIELD OF THE INVENTION

The present invention generally relates to a pharmaceutical composition for topical delivery of meloxicam. Furthermore the invention relates to a process for the manufacture of a pharmaceutical composition and to a transdermal delivery system comprising said pharmaceutical composition. In addition, the invention relates to the use of said pharmaceutical composition for the manufacture of said transdermal delivery system and to the use of an amine and/or an amide as a skin permeation enhancing agent in a pharmaceutical composition. The invention also relates to a method for treating, preventing and/or relieving the signs and/or symptoms of rheumatoid arthritis, cervico-omo-brachial syndrome, low back pain, osteoarthritis, periarthritis scapulohumeralis, tendovaginitis, peritendinitis, humerus epicondylitis, including tennis elbow, myalgia, post-traumatic tumor and pain.

### BACKGROUND OF THE INVENTION

The transdermal therapeutic administration of non-steroidal-anti-inflammatory drugs (NSAID) and corresponding pharmaceutical compositions for their topical delivery, such as creams, ointments, lotions and transdermal delivery systems, like plasters and cataplasms, are proposed in the patent literature.

In U.S. Pat. No. 6,207,184 a hydrophilic mass containing a copolymer of an aminoalkyl (meth)acrylate and alkyl (meth)acrylate is described. To the hydrophilic mass skin penetration enhancers, crosslinking agents and other ingredients may be added. As skin penetration enhancers fatty acids, their esters, alcohols, surfactants, organic bases, organic acids vitamins and lecithin are listed. Several classes of active ingredients, which may be contained in the hydrophilic mass, are named. One class consists of analgesic antiphlogistics, wherein meloxicam is named among many others.

The object of WO 02/17923 is related to a pharmaceutical composition for topical delivery comprising cyclooxygenase-2 enzyme (COX-2) inhibitor, a gelling agent, a solubilizing agent and optionally a pH modifying agent and/or other pharmaceutically acceptable adjuvants. Meloxicam is named among many other cyclooxygenase-2 enzyme inhibitors, whereby celecoxib and rofecoxib are preferred. The gelling agent may be selected from the group comprising cellulose ethers, vinyl alcohols, vinyl pyrrolidones, natural gums and acrylic polymers. The solubilizing agent which aids in the solubility and better penetration of the drug through the skin may be a volatile or a non-volatile solubilizing agent, such as alkanols and/or glycols. Furthermore the composition may contain an inorganic or organic base for modifying the pH, whereby suitable bases, e.g., alkanolamines among others, are listed. Further ingredients of the composition may be humectants, moisturizers and/or penetration enhancer, for example such as terpenes, terpene alcohols, essential oils or surfactants. Preferably the composition is a gel, spray, an aerosol, a lotion, a cream or an ointment.

A topical composition, for example an ointment, is described in JP 10324621. The composition comprises an oxicam anti-inflammatory drug, hydroxyalkyl amines, higher alcohols, carboxyvinyl polymers, lower alcohols and water.

Anti-inflammatory and analgesic patches containing styrene-isoprene-styrene block copolymer, crotamiton and a pharmaceutically active compound are described in JP 04321624.

Furthermore the document WO 01/52897 describes anti-inflammatory and analgesic compositions for topical or transdermal use which comprise a selective COX-2 inhibitor and a percutaneous absorption enhancing vehicle base. Said base comprises a percutaneous enhancer, a surfactant and a gelling or thickening agent. As percutaneous enhancer many classes of compounds are listed, for example fatty acids, alcohols, sulfoxides, amides, pyrrolidones and others.

A pharmaceutical composition for topical application comprising diclofenac and an N,N-dialkyl-alkanoylamide is described in U.S. Pat. No. 4,999,379. Such a composition is suitable for the preparations of transdermal therapeutic systems, creams, ointments, etc.

The pharmaceutical compositions comprising meloxicam show a non-satisfying percutaneous absorption behavior.

### DISCLOSURE OF THE INVENTION

The principal object of this invention is to provide a pharmaceutical composition for topical delivery of meloxicam.

One aim of this invention is to provide a pharmaceutical composition which possesses an enhanced percutaneous absorbability of meloxicam and which is especially suited to achieve therapeutic levels of meloxicam in target internal tissues.

A further object of this invention is a process for the manufacture of a pharmaceutical composition for topical delivery of meloxicam which provide enhanced skin penetration.

In addition this invention has the object to provide a transdermal delivery system for improved topical delivery of meloxicam.

Accordingly, a further object of this invention is the use of such a pharmaceutical composition for the manufacture of said transdermal delivery system.

Furthermore, this invention has the object to provide compounds as skin permeation enhancing agents of meloxicam.

A further object of this invention is to provide a method for treating, preventing and/or relieving the signs and/or symptoms of rheumatoid arthritis, cervico-omo-brachial syndrome, low back pain, osteoarthritis, periarthritis scapulohumeralis, tendovaginitis, peritendinitis, humerus epicondylitis, including tennis elbow, myalgia, post-traumatic tumor, and pain.

Further objects of this invention emanate for the ones skilled in the art from the introduction above and the following description and examples.

The inventors have found that in a transdermal application of meloxicam the absorbabililty as well as the permeation rate can be substantially improved by using an amine and/or an amide of the formulae as specified in the following as skin permeation enhancing agents. Thus pharmaceutical compositions for topical delivery of meloxicam show a considerably increased permeation rate of meloxicam when said amine and/or amide are added compared with known composition comprising conventionally used percutaneous absorption enhancer. In addition meloxicam does not tend to form crystals in the composition of this invention. Therefore these compositions are especially suited for a topical administration, including their usage in transdermal delivery systems.

Therefore the present invention relates to a pharmaceutical composition for topical delivery comprising:
(a) a pharmaceutically effective amount of meloxicam, or a pharmaceutically acceptable salt thereof;
(b) at least one skin permeation enhancing agent selected from the group consisting of: an amine of the formula I 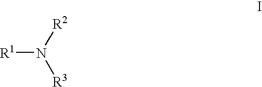
   wherein:
R¹, R², and R³ are independently of each other an alkanolyl residue with 1 to 24 C-atoms, in which one or more H-atoms may be substituted by —OH and/or one or more —CH₂— groups may be substituted by —O—, or a polyoxyethylene residue with 2 to 30 ethylene oxide units,
R² and R³ may also be independently of each other H or linear, branched or cyclic alkyl with 1 to 24 C-atoms, in which one or more —CH₂— groups may be substituted by —CH═CH— and/or —O— and/or in which one or more H-atoms may be substituted by —OH,
and an amide of the formula II 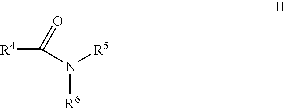
   wherein
R⁴, R⁵, and R⁶ are independently of each other a linear, branched or cyclic alkyl with 1 to 24 C-atoms, in which one or more —CH₂— groups may be substituted by —CH═CH— and/or —O— and/or in which one or more H-atoms may be substituted by —OH,
R⁶ may also be H; and
(c) at least one inert carrier.

Consequently, this invention also relates to the use of an amine of the formula I, as defined above, and/or of an amide of the formula II, as defined above, as a skin permeation enhancing agent in a concentration which enhances the skin permeability of meloxicam in a pharmaceutical composition for topical delivery.

In addition, the present invention is related to a process for the manufacture of a pharmaceutical composition for topical delivery which comprises mixing together 0.005 to 10 weight-% of said composition of meloxicam, or a pharmaceutically acceptable salt thereof, with 0.05 to 20 weight-% of said composition of at least one permeation enhancer selected from the group consisting of an amine of the formula I and an amide of the formula II according to the beforementioned definition.

Furthermore, the inventors have found that said pharmaceutical composition can advantageously be applied to a host by a transdermal delivery system.

Therefore, the present invention is also related to the use of a pharmaceutical composition according to this invention for the manufacture of a transdermal delivery system, preferably of a matrix system and/or a liquid reservoir system, most preferably of a plaster and/or a cataplasm. Consequently, this invention is further related to a transdermal delivery system comprising at least one pharmaceutical composition according to this invention.

Thus the present invention relates to a method for treating, preventing and/or relieving the signs and/or symptoms of rheumatoid arthritis, cervico-omo-brachial syndrome, low back pain, osteoarthritis, periarthritis scapulohumeralis, tendovaginitis, peritendinitis, humerus epicondylitis, including tennis elbow, myalgia, post-traumatic tumor and pain which comprises administering an effective amount of a pharmaceutical composition according to this invention and/or of a transdermal delivery system according to this invention to a host in need thereof.

There has been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying examples and claims, or may be learned by the practice of the invention.

Definition of Terms

In describing and claiming the present invention, the following terminology will be used.

The singular forms “a”, “an” and “the” include plural referents unless the context clearly indicates otherwise.

As used herein, the term “meloxicam” comprises meloxicam and pharmaceutically acceptable salts thereof.

As used herein, “transdermal delivery system” or “transdermal formulation” refer to any meloxicam containing device, system, product, chemical combination, or mechanism capable of being applied to, or against the skin, to effect transdermal delivery, of meloxicam.

As used herein, the term “skin” refers to any membrane of the human body to which a chemical formulation or composition may be applied including the external skin of the body, the mucosa membranes of the nasal, oral, vaginal, and rectal cavities.

As used herein, the term “transdermal” or “percutaneous” delivery means delivery of a substance or agent, by passage into and through the skin. Hence the terms “transdermal” and “transmucosal” are used interchangeably unless specifically stated otherwise. Likewise, the terms “skin”, “derma”, “epidermis”, “mucosa”, and the like shall also be used interchangeably unless specifically stated otherwise.

As used herein, the terms “enhancement”, “penetration enhancement”, or “permeation enhancement” refer to an increase in the permeability of the skin, to a delivery substance or agent, so as to increase the rate at which the delivery substance permeates through the skin. “Permeation enhancer”, “permeation enhancing agent”, “penetration enhancer”, or similar terms refer to a material, or materials that achieve or facilitate such permeation enhancement, and an “effective amount” of an enhancer means an amount effective to enhance penetration through the skin, of meloxicam, to a selected degree. Enhanced penetration as affected through the use of such enhancers can be observed, for example, by measuring the rate of diffusion of the delivery substance through animal or human skin using a diffusion cell apparatus.

As used herein, “effective amount” refers to the minimal amount of a substance or agent, which is sufficient to achieve a desire therapeutic effect. Therefore, when used in connection with meloxicam, effective amount connotates an amount of such agent, which is sufficient to achieve a desired meloxicam plasma level. Such plasma levels may be achieved within and sustained for various time intervals as determined by the parameters of each particular formulation. The type and amount of meloxicam, the type and amount of inert carrier, the size of the transdermal formulation, as well as the presence and amount of specific penetration enhancers may all be adjusted to arrive at a formulation which achieves the desired blood levels within a specific time interval. One of ordinary skill in the transdermal arts would be able to readily determine the amount and type of each component in the combination, which are required to achieve the target blood levels within a specified time frame.

By the term “matrix patch” or “matrix system” is meant a predetermined amount of a pharmaceutical composition according to the invention comprising a polymeric carrier or phase in which meloxicam and the skin permeation enhancing agent are dissolved or suspended. In one aspect the polymeric carrier or phase is a pressure-sensitive adhesive, whereby such a matrix system is commonly named “plaster”. The definition of a matrix system is meant to include embodiments wherein such polymeric phase is laminated to a pressure sensitive adhesive or used within an overlay adhesive to form an adhesive matrix patch with a reservoir. A matrix system usually and preferably comprises an adhesive layer having an impermeable film backing laminated onto the distal surface thereof and, before transdermal application, a release liner on the proximal surface of the adhesive. The film backing protects the polymeric phase of the matrix patch and prevents release of the delivery substance and/or enhancer to the environment. The release liner function similarly to the impermeable backing, but is removed from the matrix patch prior to application of the patch to the skin as defined above. Matrix patches are known in the art of transdermal delivery systems to routinely contain such backing and release liner components, and matrix patches according to the present invention should be considered to comprise such backing and release liner or their functional equivalents. A matrix system therefore is a unit dosage form, or type of formulation, which includes a predetermined amount of a pharmaceutical composition according to this invention comprising a polymeric carrier. Examples without limitation, of adhesive matrix transdermal patches are those described or referred to in WO 95/02404 and WO 89/07951 (relating acryloyl system), and WO 93/04677, WO 96/08245, and WO 95/03764 (relating to rubber system) which are incorporated by reference in their entirety.

As used herein, “liquid reservoir system,” its acronym “LRS,” refers to a transdermal delivery patch or system, in which the pharmaceutical composition according to this invention additionally comprises at least one carrier vehicle. In one aspect of this invention the LRS is a “liquid reservoir patch”, comprising a carrier vehicle, an impermeable backing and a skin contacting permeable membrane, or adhesive. The carrier vehicle comprises a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. In another aspect of this invention the LRS is a “cataplasm”, comprising an adhesive layer having a support laminated onto the distal surface thereof and a release liner on the proximal surface of the adhesive to be removed before transdermal application. In the same way as conventional cataplasm, cataplasm of the present invention may be prepared by coating the support with adhesive which comprises the essential components, preferably being purified water, which is the moisture adjustment solubilizer, and the pharmaceutical composition according to this invention, and the hydrophilic base. Further compounds compounded into these essential components may be volatile or non-volatile solubilizing agent, pH modifying agent, humectant, moisturizer, preservative, opacifier, fragrance, color additive, counter-irritant, inorganic fillers and/or cross linking agents, etc. as necessary. LRS patches are known in the art of transdermal drug delivery. Examples without limitation, of LRS transdermal patches are those described or referred to in U.S. Pat. Nos. 4,983,395 and 4,849,224 (relating to LRS), and WO 2001/13915, WO 2000/02563 and U.S. Pat. No. 6,039,971 (relating to patches), which are incorporated by reference in their entirety.

As used herein, “inert carrier” refers to a polymeric carrier, or other carrier vehicle into which meloxicam may be admixed in order to form a transdermal delivery formulation. Inert carriers must generally be pharmaceutically acceptable, in that they are suitable for administration to the skin without causing significant instances of adverse results. Further, inert carriers must not react with the active substance to substantially degrade it, or otherwise form impurities, which may be delivered to the skin.

As used herein, “topical formulation” refers to a chemical formulation in which meloxicam may be incorporated, which is capable of being applied directly to the skin, and which does not include supporting structures such as backing films, etc. Examples of topical formulations without limitation include, gels, aerosols, creams, lotions, pastes, ointments, etc.

The term “host” refers to mammals, for example humans, cats, dogs, cattle, sheep, horses and pigs, whereby the meaning humans is preferred.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.1 to 200 ng/mL should be interpreted to include not only the explicitly recited concentration limits of 0.1 ng/mL and 200 ng/mL, but also to include individual concentrations within that range, such as 0.5 ng/mL, 1.0 ng/mL, 5 ng/mL, 8 ng/mL, 20 ng/mL, 75 ng/mL, 120 ng/mL, 150 ng/mL, 180 ng/mL, and sub-ranges such as 0.1-10 ng/mL, 0.5-75 ng/mL, 1-50 ng/mL, 1-125 ng/mL, 5-200 ng/mL, and 60-200 ng/mL, etc. This interpretation should apply regardless of the breadth of the range or the characteristic being described.

It is to be understood that this invention is not limited to the particular compounds, compositions, materials, devices and process steps disclosed herein, but is extended to equivalents thereof as would be recognized by those ordinarily skilled in the relevant arts. It should also be understood that terminology employed herein is used for the purpose of describing particular embodiments only and is not intended to be limiting.

The pharmaceutical composition according to this invention comprises meloxicam, or a pharmaceutically acceptable salt thereof.

Pharmaceutically acceptable salts are preferably the salts of the meloxicam with an inorganic or an organic base. Suitable salts with an inorganic base are for example the sodium, potassium or ammonium salt of meloxicam. Examples of salts with an organic base are the meglumine salt, the tris-salt or a salt of meloxicam with a basic amino acid, such as L-lysine or L-arginine.

According to the definition of the amine of the formula I and of the amide of the formula II a —CH₂— group in an alkanoyl- or an alkyl-residue may be substituted by —O—. It is obvious for a person skilled in the art that this definition does not encompass alkanoyl- or alkyl-residues wherein two or more O-atoms are directly linked to each other.

According to a first aspect of this invention those amines of the formula I are preferred as skin permeation enhancing agents wherein R¹, R², and R³ are independently of each other an alkanolyl residue with 2 to 8 C-atoms and R³ may also be H.

The alkanolyl residue may be linear, branched or cyclic and has preferably 2, 3, 4, 5 or 6 C-atoms. Preferred alkanolyl residues are ethanolyl (ethan-2-ol-1-yl), isopropanolyl (propan-2-ol-1-yl), propanolyl (ethan-3-ol-1-yl), and butanolyl (butan-4-ol-1-yl).

Particularly preferred amines are selected from the group consisting of ethanolamine, diethanolamine, triethanolamine, propanolamine, diisopropanolamine, triisopropanolamine, butanolamine, dibutanolamine, and tributanolamine.

According to a second aspect of this invention those amines of the formula I are preferred as skin permeation enhancing agents wherein:
R¹ is a polyoxyethylene residue with 2 to 30 ethylene oxide units, and
R² and R³ are independently of each other a linear, branched or cyclic alkyl residue with 1 to 24 C-atoms, in which one or more —CH₂— groups may be substituted by —CH═CH— and/or —O— and/or in which one or more H-atoms may be substituted by —OH, and
R³ may also be H.

The polyoxyethylene residue preferably consists of 2 to 20, most preferably of 5 to 20 ethylene oxide units.

Preferably R² and R³ are independently of each other an alkyl residue with 6 to 22 C-atoms in which 1, 2, 3, or 4 —CH₂— groups may be substituted by —CH═CH—. The alkyl residue may be linear, branched or cyclic. Preferably the alkyl residue is linear. R² and/or R³ have preferably the meaning of saturated or unsaturated residues of fatty acids. Thus preferred meanings of R² and/or R³ are oleyl, stearyl, myristyl, lauryl.

R³ has the meaning as defined above or may be H. Most preferably R³ is H.

Examples of preferred amines according to the second aspect of this invention are selected from the group consisting of polyoxyethylene oleylamine, polyoxyethylene stearylamine, polyoxyethylene myristylamine, polyoxyethylene laurylamine wherein the polyoxyethylene group consists of 2 to 30, preferably of 5 to 20 ethylene oxide units.

According to a third aspect of this invention the amides of the formula II are preferred as skin permeation enhancing agents wherein
R⁴ is a linear, branched or cyclic alkyl residue with 4 to 20 C-atoms, in which one or more —CH₂— groups may be substituted by —CH═CH— and/or —O— and/or in which one or more H-atoms may be substituted by —OH,
R⁵ and R⁶ are independently of each other an alkanolyl with 2 to 8 C-atoms, and
R⁶ may also be H.

Preferably R⁴ is an alkyl residue with 6 to 22 C-atoms in which 1, 2, 3, or 4 —CH₂— groups may be substituted by —CH═CH—. The alkyl residue may be linear, branched or cyclic. Preferably the alkyl residue is linear. R⁴ has preferably the meaning of a saturated or unsaturated residue of a fatty acid. Thus preferred meanings of R⁴ are myristyl and lauryl.

R⁵ and R⁶ are independently of each other an alkanolyl residue which may be linear, branched or cyclic and has preferably 2, 3, 4, 5 or 6 C-atoms. Preferred alkanolyl residues are ethanolyl (ethan-2-ol-1-yl) and isopropanolyl (propan-2-ol-1-yl).

Preferred amides according to this invention are selected from the group consisting of coconut fatty acid diethanolamide and lauric fatty acid diethanolamide.

The pharmaceutical composition according to this invention may comprise one skin permeation enhancing agent according to the first, second, and third aspect of this invention alone or in combination with one or more skin permeation enhancing agents according to this invention and/or in combination with one or more skin permeation enhancing agents known to the one skilled in the arts.

The content of meloxicam is preferably from 0.005 to 10 weight-% of said composition and of the amine and/or amide permeation enhancing agent from 0.05 to 20 weight-% of said composition. Particularly preferred ranges are of meloxicam 0.05 to 8 weight-% and of the amine and/or amide as permeation enhancing agent from 0.1 to 10 weight-% related to the total composition.

According to a preferred embodiment of this invention, the composition comprises meloxicam in a range of 0.1 to 5 weight-%, more preferably from 0.5 to 2.5 weight-%, of said composition and at least one amine of the formula I as a permeation enhancing agent in a range from 1 to 15 weight-%, more preferably from 3 to 10 weight-%, related to the total composition.

Furthermore the inventors found that the combination of said amine and/or amide permeation enhancing agent with at least one compound selected from the group consisting of terpenes, terpene alcohols, fatty acids, fatty acid esters and fatty alcohols results in a further enhancement of the permeation of meloxicam through the skin. For convenience in the following the term “further permeation enhancer” is used for the compounds of this group.

The term terpene includes terpenoid compounds. Preferred terpenes or terpene alcohols are selected from the group consisting of 1-menthol, eucalyptus oil, mentha oil, cineole, and limonene.

Preferred fatty acid have 8 to 20 C-atoms. Examples of preferred fatty acids are oleic acid, alkanoic acids, capric acid, palmitic acid, myristic acid, hexanoic acid, lactic acid, lauric acid, linoleic acid, stearic acid, isostearic acid, and mixtures thereof. Particularly preferred fatty acids are capric acid, oleic acid, palmitic acid, lauric acid, myristic acid, stearic acid, and isostearic acid.

Preferred fatty acid esters are the esterification products of a fatty acid with 8 to 20 C-atoms and an alcohol with 1 to 12 C-atoms. Examples of suitable fatty acid esters are methyl laurate, glycerol monooleate (GMO), sorbitan monooleate (SMO), glycerol monolaurate (GML), glycerol monolinoleate (GMLO), isopropyl myristate, isopropyl palmitate, methyl propionate, monoglycerides, propylene glycol monolaurate, sorbitan monolaurate, diisopropyl adipate, and mixtures thereof. Acceptable fatty acid esters of lactic acid or glycolic acid or their salts include but are not limited to lauroyl glycolate, sodium lauryol glycolate, caproyl glycolate, sodium caproyl glycolate, cocyl glycolate, sodium cocyl glycolate, isostearoyl glycolate, tromethamine lauroyl glycolate, lauroyl lactylate, sodium lauroyl lactylate, caproyl lactylate, sodium caproyl lactylate, cocoyl lactylate, sodium cocyl lactylate, isostearoyl lactylate, tromethamine lauryol lactylate, and mixtures thereof. Particularly preferred fatty acid esters are diisopropyl adipate and isopropyl myristate.

Preferred fatty alcohols as further permeation enhancer have 8 to 20 C-atoms and may possess 1, 2 or 3 C—C double bonds. Examples of preferred fatty alcohols are lauryl alcohol, caprylic alcohol, myristyl alcohol, cetyl alcohol, stearyl alcohol, aliphatic alcohols, linolenyl alcohol, nerolidol, oleyl alcohol, and mixtures thereof. Particularly preferred fatty alcohols are lauryl alcohol, oleyl alcohol, stearyl alcohol, caprylic alcohol, and myristyl alcohol.

The content of said further permeation enhancer is preferably from 0.05 weight-% to 60 weight-% related to the total composition. A particularly preferred lower limit is 0.5 weight-%, most preferably 2 weight-%; and a particularly preferred upper limit is 50 weight-%, most preferably 40 weight-%, very most preferably 30 weight-%.

A preferred pharmaceutical composition for topical delivery comprises:
(a) a pharmaceutically effective amount of meloxicam, or a pharmaceutically acceptable salt thereof,
(b) at least one skin permeation enhancing agent according to this invention, preferably at least one amine of the formula (I); and
(c) one, two, three, or more further permeation enhancers selected from terpenes, terpene alcohols, fatty acids, fatty acid esters and fatty alcohols,
   preferably at least one further permeation enhancer selected from terpenes and terpene alcohols, and one, two or more further permeation enhancer selected from fatty acids, fatty acid esters and fatty alcohols;
   more preferably at least one further permeation enhancer selected from terpenes and terpene alcohols, and at least one further permeation enhancers selected from fatty acids, and at least one further permeation enhancer selected from fatty acid esters.

In the before-mentioned preferred pharmaceutical composition, the preferred weight ratios of component (a):component (b):component (c) is preferably in a range of 0.1 to 5.0:1 to 15:10 to 50.

A particularly preferred pharmaceutical composition for topical delivery comprises:
(a) a pharmaceutically effective amount of meloxicam, or a pharmaceutically acceptable salt thereof;
(b) at least one amine of the formula (I) as a permeation enhancer, preferably diisopropanolamine and/or triisopropanolamine; and
(c) at least one further permeation enhancer selected from terpenes and terpene alcohols, and one, two or more further permeation enhancers selected from fatty acids, fatty acid esters and fatty alcohols,
   preferably at least one further permeation enhancer selected from terpenes and terpene alcohols, and at least one further permeation enhancer selected from fatty acids, and at least one further permeation enhancer selected from fatty acid esters.

In the before-mentioned preferred pharmaceutical composition, the preferred weight ratios of component (a):component (b):component (c) is preferably in a range of 0.1 to 5.0:1 to 15:10 to 50.

A particularly preferred pharmaceutical composition for topical delivery comprises:
(a) a pharmaceutically effective amount of meloxicam, or a pharmaceutically acceptable salt thereof;
(b) one or two permeation enhancers selected from the group consisting of diisopropanolamine and triisopropanolamine; and
(c) at least one further permeation enhancer selected from terpenes and terpene alcohols, preferably 1-menthol, and at least one further permeation enhancer selected from fatty acids, preferably stearic acid or isostearic acid, and at least one further permeation enhancer selected from fatty acid esters, preferably isopropyl myristate.

In the before-mentioned preferred pharmaceutical composition, the preferred weight ratios of component (a):component (b):component (c) is preferably in a range of 0.1 to 5.0:1 to 15:10 to 50.

A most preferred pharmaceutical composition for topical delivery comprises:
(a) a pharmaceutically effective amount of meloxicam, or a pharmaceutically acceptable salt thereof;
(b) one or two permeation enhancers selected from the group consisting of diisopropanolamine and triisopropanolamine;
(c1) 1-menthol as a first further permeation enhancer;
(c2) isopropyl myristate as a second further permeation enhancer; and
(c3) isostearic acid and/or stearic acid as a third further permeation enhancer.

In the before-mentioned preferred pharmaceutical composition, the preferred weight ratios of component (a):component (b):the sum of components (c1), (c2), and (c3) is preferably in a range of 0.1 to 5.0:1 to 15:10 to 50. More specifically, the preferred weight ratios of component (a):component (b):components (c1):component (c2):component (c3) is preferably in a range of 0.5 to 2.5:3 to 10:2 to 4:10 to 30:3 to 5.

The pharmaceutical composition according to this invention preferably comprises at least one adhesive, gelling and/or thickening agent. Preferred representatives are described in the following sections on transdermal formulations and transdermal delivery systems.

A preferred content of the adhesive, gelling and/or thickening agent is within the range from 1 to 99 weight-% of said composition. A preferred lower limit is 5 weight-%, a preferred upper limit is 97 weight-%.

The pharmaceutical composition according to this invention may additionally comprise at least one agent selected from the group consisting of volatile or non-volatile solubilizing agent, pH modifying agent, humectant, moisturizer, preservative, opacifier, fragrances, color additives, and counter-irritants.

In addition to a pharmaceutical composition and a transdermal delivery system, the present invention relates to a method for treating, preventing and/or relieving the signs and/or symptoms of rheumatoid arthritis, cervico-omo-brachial syndrome, low back pain, osteoarthritis, periarthritis scapulohumeralis, tendovaginitis, peritendinitis, humerus epicondylitis, including tennis elbow, myalgia, post-traumatic tumor and pain.

In one aspect, the desired blood plasma level of meloxicam is achieved within about 0.25 to about 18 hours after initial administration of the inventive composition. In yet another aspect, the meloxicam blood plasma level is achieved within about 0.5 to about 12 hours after initiation administration of the composition according to this invention. In a further aspect, the meloxicam blood plasma level is sustained for a period of at least about 24-96 hours from a single transdermal administration.

The time frame for achieving desired plasma levels may be determined by such parameters as the type and size of the transdermal delivery system, the amount of the meloxicam present in the composition, and the skin flux rate achieved by the composition. Further, the flux rate is determined by the type and amount of the one or more skin permeation enhancing agents and of the optionally one or more further permeation enhancers. Elements such as patch size, meloxicam content and concentration, enhancer amount, and enhancer type may all be coordinated in order to achieve the desired blood plasma levels within a desired amount of time, as can be readily determined by one skilled in the art. Others physiological factors, such as variations in individual skin type and permeability may effect the ultimate meloxicam blood plasma level and the time frame in which it is achieved.

In one aspect, permeation rates of the meloxicam through living human skin may be in the range of about 0.025 μg/cm²/hr to about 50 μg/cm²/hr. A preferred lower limit of the permeation rate is 0.05 μg/cm²/hr, particularly 0.1 μg/cm²/hr.

In a further aspect, the transdermal formulation may have a size of from about 1 to 200 cm², preferably from about 5 to 100 cm².

However, these general parameters are not limitations on the way in which the desired blood serum levels may be achieved. Different permeation rates, times, and amounts may be used to effect the desired blood levels by employing a formulation which uses different parameters.

Furthermore, the pharmaceutical composition according to this invention may comprise further positive health benefit conferring substances, or treatment agents.

In one aspect, the pharmaceutical composition of the present invention may be a topical formulation. As recited above, topical formulations may take a variety of specific forms, such as gels, ointments, pastes, mousses, aerosols, creams, lotions, and other hydrophobic or water-miscible vehicles. Such topical formulations usually comprise at least one hydrophobic agent, water-miscible agent, emulsifier and/or thickener. Other specific types of topical formulations not specifically mentioned will be readily recognized by those skilled in the art and fall within the purview of the present invention.

Specific examples of suitable hydrophobic and water-miscible agents include but are not limited, hydrocarbons (e.g., liquid paraffin, mineral oil, paraffin oil, white petrolatum, squalane), silicones (e.g., liquid polymethylsilaxanes, dimethicone), alcohols (e.g., ethanol, isopropyl alcohol, lauryl alcohol), polyols and polyglycols (e.g., propyl glycol, glycerin, triacetin, polyethylene glycols), sterols (e.g., lanolin, cholesterol), carboxylic acids (e.g., lauric acid, oleic acid), esters and polyesters (e.g., ethylene glycol monostearate, sorbitan monoesters, glyceryl tristearate, olive oil, soybean oil, isopropyl myristate, isopropyl palmitate).

Specific examples of suitable emulsifiers include, but are not limited to sterols and sterol eaters (e.g., cholesterol), carboxylic acid salts (sodium, ethanol amine, etc. of lauric acid, oleic acid, etc.), esters and polyesters (e.g., ethylene glycol monoesters, propylene glycol monoesters, glycerol monoesters, sorbitan monoesters, sorbitol monoesters, polyoxyethylene esters, sorbitan diesters, polyoxyethylene sorbitan polyesters—tweens), ethers and polyethers (e.g., polyethylene glycol monocetyl ethers, polyethylene-polypropylene glycols—pluronics), others (e.g., sodium lauryl sulfate, borax, ethanolamine).

Specific examples of suitable thickeners include, but are not limited to acrylate copolymers, algin, behenyl alcohol, 18-36 acid triglycerides, calcium carboxymethyl cellulose, PVP/MA copolymers, carbomer (910, 934, 934p, 940, 941, 1342, etc.), carboxymethylcellulose sodium, cellulose, cetyl alcohol, guar gum, hydroxyethylcellulose, hydroxypropylcellulose, hydroxypropylmethyl-cellulose, methylcellulose, methyl hydroxyethylcellulose, PEGs, poloxamine (304, 504, 701, 904, 1102, 1304, 1502, etc.), polycarbophil, polyethylene, propylene glycol alginate, PVP, PVPNA copolymer, silica, silicones, aluminum hydroxide gel, beeswax.

Preferably the formulation for application in a liquid reservoir system, especially a cataplasm, additionally contains phosphoric and/or tartaric acid which may serve as pH modifying agent or solubilizing agent. Especially, tartaric acid dissolves gradually dried aluminum hydroxide gel in the cataplasm. Other acids, such as glycolic acid, lactic acid, malic acid, gluconic acid, salicylic acid, are also suitable agents.

The pharmaceutical composition of the present invention may be incorporated in a transdermal delivery system in order to provide a transdermal meloxicam delivery system. Such a transdermal delivery system may either be a matrix system, for example an adhesive matrix patch or a plaster, or a liquid reservoir system, for example a liquid reservoir patch or a cataplasm, or the like.

In the case of an adhesive matrix patch or a plaster, a type and amount of a pharmaceutical composition according to this invention sufficient to produce the desired therapeutic blood plasma level of meloxicam is dissolved or suspended in a polymeric phase or carrier. One or more further permeation enhancers may be included in the polymeric phase, as well as additional positive health benefit imparting substances. The size of an adhesive matrix patch or plaster may be adjusted to provide varying dosage amounts, and may vary from about 1 to 200 cm². In another aspect, the size of an adhesive matrix patch may be from about 5 to about 100 cm².

A wide range of adhesives useful in connection with transdermal patches will be known to those skilled in the art of transdermal drug delivery. In one aspect of the invention, acceptable adhesives may include polyacrylate polymers, rubber-based adhesives, and polysiloxanes adhesives.

In one aspect, polyacrylate polymers can be any of the homopolymers, copolymers, block copolymer, terpolymers, and the like of various acrylic acids. In another aspect of the invention, the acrylate polymers may be a combination of one or more monomers of acrylic acids and other copolymerizable monomers.

Acrylate polymers may also include copolymers of alkyl acrylates and/or methacrylates, and/or copolymerizable secondary monomers or monomers with functional groups. Specific examples of acrylate monomers, which are suitable for use with the present invention include, but are not limited to methacrylic acid, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecylmethacrylate, tridecyl acrylate, tridecyl methacrylate, and mixtures thereof.

Specific examples of functional monomers which are copolymerizable with the above-recited alkyl acrylates or methacrylates, which can also be used include, but are not limited to acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tertbutylaminoethyl acrylate, tert-butylaminoethyl methacrylate, methoxyethyl acrylate, methoxyethyl methacrylate, and mixtures thereof.

Especially suitable acrylic copolymers are acrylic ester-vinyl acetate copolymers, methacrylic acid-n-butyl acrylate copolymers and methyl methacrylate-2-ethylhexyl acrylate copolymers. These polymers are commercially available as Nissetsu PE300, Ultrazol W-51CL and Nikazol TS-620, respectively.

In one aspect, utilizing a mixture of two or more acrylic polymers may facilitate sustained release of the meloxicam. Many variations and combinations of acrylics may be employed to achieve the desired increase in release duration.

Specific examples of suitable rubber-based pressure sensitive adhesives include, but are not limited to hydrocarbon polymers, such as natural and synthetic polyisoprenes, polybutylenes and polyisobutylene (PIB), styrene/butadiene polymers, styrene-isoprene-styrene block copolymers, hydrocarbon polymers such as butyl rubber, halogen-containing polymers such as polyacrylic nitrile, polytetrafluoroethylene, polyvinyl chloride, polyvinylidene chloride, and polychlorodiene, and polysiloxanes, and other copolymers thereof.

Specific examples of suitable polysiloxanes include but are not limited to silicone pressure sensitive adhesives, which are based on two major components: a polymer, or gum, and a tackifying resin. The rosin resin is a preferred representative of the tackifying resin. The polysiloxane adhesive may be prepared by cross-linking the gum, typically a high molecular weight polydiorganosiloxane with the resin to produce a three-dimensional silicate structure via a condensation reaction in an appropriate organic solvent.

A particularly preferred transdermal delivery system according to this invention comprises
(a) a pharmaceutically effective amount of meloxicam, or a pharmaceutically acceptable salt thereof;
(b) one or two permeation enhancers selected from the group consisting of diisopropanolamine and triisopropanolamine;
(c1) 1-menthol as a first further permeation enhancer;
(c2) isopropyl myristate as a second further permeation enhancer;
(c3) isostearic acid and/or stearic acid as a third further permeation enhancer; and
(d) an acrylic copolymer, preferably an acrylic block copolymer, preferably being crosslinked, for example by a crosslinking agent like adipic acid dihydrazide.

In the before-mentioned preferred transdermal delivery system, the preferred amounts of the individual components are as follows: (a) 0.5 to 2.5 weight-%, (b) 3 to 10 weight-%, (c1) 2 to 4 weight-%, (c2) 10 to 30 weight-%, (c3) 3 to 5 weight-%, and (d) 50 to 90 weight-%, wherein weight-% is related to the weight of the total composition.

In use, the matrix patch contains a distal backing and a proximal release liner laminated on the polymer layer. The distal backing defines the side of the matrix patch that faces the environment, (i.e., distal to the skin or mucosa), and the release liner is adhered to the proximal side and must be removed before patch application. The backing layer functions to protect the matrix polymer layer with the delivery substances and enhancer, and to provide an impenetrable layer that prevents loss of delivery substance to the environment. Thus, the material chosen for the backing should be compatible with the polymer layer, delivery substances, and enhancer, and should be minimally permeable to any components of the matrix patch.

Advantageously, the backing can be opaque to protect components of the matrix patch from degradation caused by exposure to ultraviolet light. Further, the backing should be capable of binding to and supporting the polymer layer, yet should be pliable to accommodate the movements of a person using the matrix patch. Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene. A thickness of about 0.01 to about 0.3 mm may be preferred. The release liner can be made of the same materials as the backing, or other suitable films coated with an appropriate release surface.

The matrix patch can further comprise various additives in addition to the polymer layer, delivery substance, and permeation enhancer that are preferably the fundamental components of the adhesive matrix patch formulation. These additives are generally those pharmaceutically acceptable ingredients that are known in the art of transdermal substance delivery. However, such additive ingredients must not materially alter the basic and novel characteristics of the matrix patch. For example, suitable diluents can include mineral oil, low molecular weight polymers, plasticizers, and the like.

Many transdermal delivery substance formulations have a tendency to irritate the skin after prolonged exposure thereto, thus addition of a skin irritation reducing agent aids may be desirable.

The LRS patch generally contains a backing layer having a reservoir portion configured to contain the pharmaceutical composition according to this invention wherein the meloxicam and at least one skin permeation enhancing agent are admixed or dissolved in a carrier vehicle. Such carrier vehicles may be the same as those used for topical applications described above. Further, a micro- or nanoporous membrane may be heat sealed across the opening of the reservoir in order to control the rate at which meloxicam is transmitted to the skin. Additionally, an adhesive layer will generally be applied to a portion of the backing layer surrounding the reservoir for adhering the LRS patch to the skin. Further, a release liner that is removed prior to application is placed upon the adhesive to prevent adhesion of the patch prior to application. In use, the release liner is removed, and the patch is adhered to the skin at a selected application situs. When the contents of the reservoir have been depleted, the patch may be removed.

### EXAMPLES AND EXPERIMENTS

The following examples of compositions and transdermal delivery systems having a variety of meloxicam containing formulations are provided to promote a more clear understanding of the possible combinations of the present invention, and are in no way meant as a limitation thereon.

1. Study on Percutaneous Absorption Enhancer in Skin Permeability of Meloxicam in an Aqueous Composition System

The following method for studying the skin permeation of a drug was employed. Isolated skin from a hairless mouse was punched between 2-chamber diffusion cells (horizontal diffusion cell), and 0.9 mL of buffer (pH 7.4) was added to the receiver (the dermis) side, and then was stirred by a magnetic stirrer. To the donor (the stratum corneum) side, each 0.9 mL of the samples was applied, and the solution in the receiver was sampled with time, and the drug concentration was determined by high-performance liquid chromatography to calculate the amount of drug that have permeated through the skin. Percutaneous absorption enhancer shown in each table was compounded with purified water, and the excess amount of meloxicam was added to obtain a suspension, which was used as a sample for permeation test.

TABLE 1
Study on percutaneous absorption enhancer
in skin permeability of meloxicam
(Purified water was used as a solvent)

| Sample | | Flux |
|---|---|---|
| No. | Percutaneous Absorption Enhancer | μg/cm²/hr |
| 1 | Diisopropanolamine (5% by weight) | 110.5 ± 38.8 |
| 2 | Triisopropanolamine (1% by weight) | 39.9 ± 29.7 |
| 3 | Diethanolamine (1% by weight) | 24.6 ± 8.0 |
| 4 | Triethanolamine (4% by weight) | 30.2 ± 0.5 |
| 5 | Lauric acid diethanolamide (3% by weight) | 79.1 ± 10.2 |
| 6 | Coconut fatty acid diethanolamide | 41.8 ± 16.4 |
| | (4% by weight) | |
| 7 | Polyoxyethylene oleylamine (5 EO) | 15.3 ± 5.5 |
| | (1% by weight) | |
| 8 | Polyoxyethylene oleylamine (15 EO) | 14.7 ± 5.1 |
| | (1% by weight) | |
| 9 | Diisopropanolamine (5% by weight) + | 1875.8 ± 209 |
| | 1-menthol (4% by weight) | |
| 10 | Diisopropanolamine (5% by weight) + | 224.4 ± 49.9 |
| | eucalyptus oil (1% by weight) | |
| 11 | Diisopropanolamine (5% by weight) + lauryl | 330.7 ± 50.4 |
| | alcohol (3% by weight) | |
| 12 | Diisopropanolamine (5% by weight) + capric | 167.9 ± 18.1 |
| | acid (0.9% by weight) | |
| A | Control | 3.7 ± 0.0 |
| B | Crotamiton (1% by weight) | 2.5 ± 0.6 |
| C | Isopropanol (70% by weight) | 4.5 ± 2.0 |
| D | N-Methyl-2-pyrrolidone (8% by weight) | 6.0 ± 1.2 |
| E | Eucalyptus oil (5% by weight) | 4.8 ± 1.9 |
| F | Lauryl alcohol (3% by weight) | 3.2 ± 1.0 |

The letters A to F denote comparative samples, comprising comparative amine or amide skin permeation enhancers. The flux (skin permeation rate of drug) was calculated in the steady state.

Compared with the samples A to F a particular enhancement of the skin permeability of meloxicam is observed if an amine or amide compound of this invention is used. A particular advantageous skin permeation enhancing agent is diisopropanolamine. A further improvement of the skin permeation is observed when the amine is combined with 1-menthol, eucalyptus oil, lauryl alcohol or capric acid as further permeation enhancers, whereby 1-menthol in combination with diisopropanolamine is particularly preferred.

2. Study of Skin Permeability of Meloxicam From Plaster

The sample of plaster was made by the following preparation method, and each sample was applied to the donor (the stratum corneum) side of the skin to test the skin permeability of drug in the same manner as the above experimental method 1, except for the use of a horizontal diffusion cell (1.2 mL of volume).

The plaster was manufactured by the following method. In accordance with the formulation shown in table 2, meloxicam, acrylic copolymers, skin permeation enhancing agents (diisopropanolamine according to this invention or N-methyl-2-pyrroridone according to the comparative sample) and optionally 1-menthol as further permeation enhancer were mixed, and the adhesive mixture thus obtained was coated on a polyethylene film (1 g/70 cm²), dried at 70° C. for 15 minutes, and then the volatiles (volatile solvents or water) were allowed to evaporate. A polyethylene terephthalate foil as release liner was put on it to give a plaster which was cut into the desired size.

TABLE 2
Study on skin permeation enhancer in plaster
(the compounding ratio is based on by weight)

| | Sample | Sample | Sample | Sample | | | |
|---|---|---|---|---|---|---|---|
| | 13 | 14 | 15 | 16 | Sample G | Sample H | Sample I |
| Meloxicam | 1 | 1 | 1 | 1 | 1 | 1 | 1 |
| Nissetsu PE300 | 94 | | | 90 | 97.4 | | |
| Ultrazol W- | | 94 | | | | 97.2 | |
| 51CL | | | | | | | |
| Nikazol TS-620 | | | 94 | | | | 97.3 |
| Diisopropanolamine | 5 | 5 | 5 | 5 | | | |
| N-Methyl-2- | | | | | 1.6 | 1.8 | 1.7 |
| pyrrolidone | | | | | | | |
| l-Menthol | | | | 4 | | | |
| Flux | 0.187 | 0.577 | 0.446 | 0.380 | 0.020 | 0.018 | 0.007 |
| (μg/cm²/hr) | | | | | | | |
| Presence of | absent | absent | absent | absent | present | present | present |
| drug crystals | | | | | | | |

The following acrylic copolymers were used, which are commercially available from:
Nissetsu PE300: acrylic ester-vinyl acetate copolymer (solvent type) (Nippon Carbide Industries Co., Inc./Japan)
Ultrazol W-51CL: methacrylic acid-n-butyl acrylate copolymer (emulsion type) (Ganz Chemical Co., Ltd.)
Nikazol TS-620: methyl methacrylate-2-ethylhexyl acrylate copolymer (emulsion type) (Nippon Carbide Industries Co., Inc./Japan)

The use of diisopropanolamine in a plaster according to experiment 2 resulted in a great improvement of the skin permeability of meloxicam compared with N-methyl-2-pyrroridone (samples G to I). The permeation rate is approximately doubled by using 1-menthol as a further permeation enhancer. Furthermore it can be seen that the formation of meloxicam crystals is prevented by the use of diisopropanolamine, thus a superior pharmaceutical preparation can be provided according to this invention.

3. Study on Skin Permeability of Drug From Cataplasm-Type Patch

The sample of cataplasm was made by the following preparation method, and each sample was applied to the donor (the stratum corneum) side of the skin to test the skin permeability of drug in the same manner as the above experimental method 1, except for the use of a vertical diffusion cell (1.2 mL of volume).

A cataplasm was prepared according to the following procedure. To a solution prepared by dissolving or dispersing phosphoric acid, disodium edetate, and light anhydrous silicic acid in purified water is added a solution prepared by dispersing carboxymethylcellulose sodium in concentrated glycerin. Then, a solution prepared by dispersing partially neutralized polyacrylate and dried aluminum hydroxide gel in concentrated glycerin was added, and the resulting mixture was thoroughly kneaded. Subsequently, a solution prepared by mixing polyoxyethylene oleylamine, 1-menthol, and meloxicam was added, and finally a solution prepared by dissolving tartaric acid in purified water was added, and the resulting mixture was thoroughly kneaded to prepare a base. The base thus obtained was spread on a polypropylene film, and covered with a backing (a nonwoven fabric), cut into the size of 10 cm long×14 cm width, and placed in a laminated bag to obtain a cataplasm. Each composition was formulated in accordance with a column of the tables 3 and 4.

TABLE 3
Study on polyoxyethylene oleylamine as percutaneous
absorption enhancer in combination with a terpene
in a cataplasm (the compounding ratio is based on by weight)

| | Sample | Sample | Sample | Sample | Sample |
|---|---|---|---|---|---|
| | 17 | 18 | 19 | 20 | J |
| Meloxicam | 0.1 | 0.1 | 0.1 | 0.1 | 0.1 |
| l-Menthol | | 4.0 | | 4.0 | |
| Eucalyptus oil | | | 1.5 | 1.5 | |
| Polyoxyethylene | 1.0 | 1.0 | 1.0 | 1.0 | |
| oleylamine (5 EO) | | | | | |
| Phosphoric acid | 0.2 | 0.2 | 0.2 | 0.2 | 0.2 |
| Disodium edetate | 0.1 | 0.1 | 0.1 | 0.1 | 0.1 |
| Light anhydrous | 1.0 | 1.0 | 1.0 | 1.0 | 1.0 |
| silicic acid | | | | | |
| Dried aluminum | 0.05 | 0.05 | 0.07 | 0.07 | 0.05 |
| hydroxide gel | | | | | |
| Carboxymethyl- | 2.0 | 2.0 | 2.0 | 2.0 | 2.0 |
| cellulose sodium | | | | | |
| Partially neutralized | 7.0 | 7.0 | 7.0 | 7.0 | 7.0 |
| polyacrylate | | | | | |
| Tartaric acid | 1.3 | 1.3 | 1.3 | 1.3 | |
| Concentrated glycerin | 35.0 | 35.0 | 35.0 | 35.0 | 35.0 |
| Purified water | 52.25 | 48.25 | 50.73 | 46.73 | 54.55 |
| Total | 100 | 100 | 100 | 100 | 100 |
| Flux (μg/cm²/hr) | 0.056 | 0.34 | 0.28 | 0.52 | 0.002 |
| Presence of drug crystals | absent | absent | absent | absent | present |

Sample J is a comparative example.

TABLE 4
Study on polyoxyethylene oleylamine as percutaneous absorption
enhancer in combination with a fatty acid ester in a cataplasm
(the compounding ratio is based on by weight)

| | Sample 21 | Sample 22 | Sample 23 |
|---|---|---|---|
| Meloxicam | 0.1 | 0.1 | 0.1 |
| 1-Menthol | | | 4 |
| Diisopropyl adipate | | 10 | 10 |
| 10% Phosphoric acid | 0.2 | 0.2 | 0.2 |
| Disodium edetate | 0.1 | 0.1 | 0.1 |
| Light anhydrous silicic acid | 1 | 1 | 1 |
| Dried aluminum hydroxide gel | 0.05 | 0.07 | 0.07 |
| Carboxymethylcellulose sodium | 2 | 2 | 2 |
| Partially neutralized polyacrylate | 7 | 7 | 7 |
| Tartaric acid | 1.3 | 1.3 | 1.3 |
| Polyoxyethylene oleylamine | 1 | 1 | 1 |
| (5 EO) | | | |
| Sorbitan fatty acid esters | | 0.3 | 0.3 |
| Concentrated glycerin | 35 | 35 | 35 |
| Purified water | 52.25 | 41.93 | 37.93 |
| Flux (μg/cm²/hr) | 0.056 ± 0.04 | 0.12 ± 0.01 | 0.29 ± 0.1 |
| Presence of drug crystals | absent | absent | absent |

In comparison with the sample J in table 3 a great improvement in the skin permeability results in the use of an polyoxyethylene derivative of an amine (in this case of polyoxyethylene oleylamine (5 EO), i.e. with 5 ethylene oxide units) as a skin permeation enhancer. A further increase by a factor of 5 to 10 is achieved by co-compounding a terpene, in this case 1-menthol and/or eucalyptus oil. In addition the formation of meloxicam crystals is avoided by the composition of this invention.

The sample 21 of table 4 corresponds to the sample 17 of table 3. As can be seen from the flux rates of the samples 22 and 23 a great improvement is achieved by combining the polyoxyethylene amine with a fatty acid ester, in this case diisopropyl adipate, and optionally with 1-menthol as further permeation enhancers. The compositions according to these samples also do not form any drug crystals.

4. Example of a Plaster

An example of a pharmaceutical composition according to this invention is characterized by the following formulation:

| | |
|---|---|
| Styrene-isoprene-styrene block copolymer | 25 parts by weight |
| Hydrogenated rosin resin | 25 parts by weight |
| Liquid paraffin | 44 parts by weight |
| Diisopropanolamine | 5 parts by weight |
| Meloxicam | 1 parts by weight |

The mixture obtained by heat mixing according to the above formulation was spread on a polyester film, dried and covered with a release liner to give an adhesive matrix patch of the present invention (plaster).

1. A pharmaceutical composition comprising:
(a) a pharmaceutically effective amount of meloxicam, or a pharmaceutically acceptable salt thereof;
(b) a skin permeation enhancing agent selected from the group consisting of:
(i) an amine of formula I 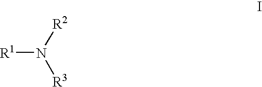
wherein:
R¹, R², and R³ are each independently an alkanolyl group with 1 to 24 C-atoms, in which one or more H-atoms are optionally substituted by —OH and/or one or more —CH₂— groups are optionally substituted by —O—, or a polyoxyethylene group with 2 to 30 ethylene oxide units, and
R² and R³ are additionally independently H or a linear, branched, or cyclic alkyl group with 1 to 24 C-atoms, in which one or more —CH₂— groups are optionally substituted by —CH═CH— and/or —O— and/or in which one or more H-atoms are optionally substituted by —OH, and
(ii) an amide of formula II 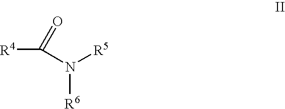
wherein:
R⁴ and R⁵ are each independently a linear, branched, or cyclic alkyl group with 1 to 24 C-atoms, in which one or more —CH₂— groups are optionally substituted by —CH═CH— and/or —O— and/or in which one or more H-atoms are optionally substituted by —OH, and
R⁶ is H or a linear, branched, or cyclic alkyl group with 1 to 24 C-atoms, in which one or more —CH₂— groups are optionally substituted by —CH═CH— and/or —O— and/or in which one or more H-atoms are optionally substituted by —OH; and
(c) at least one inert carrier.

2. The pharmaceutical composition according to claim 1, wherein the skin permeation enhancing agent is the amine of formula I 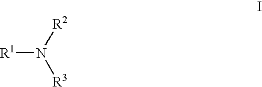
wherein:
R¹ and R² are each independently an alkanolyl group with 2 to 8 C-atoms, and
R³ is H or an alkanolyl group with 2 to 8 C-atoms.

3. The pharmaceutical composition according to claim 1, wherein the skin permeation enhancing agent is the amine of formula I 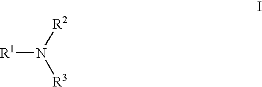
wherein:
R¹ is a polyoxyethylene group with 2 to 30 ethylene oxide units,
R² is a linear, branched, or cyclic alkyl group with 1 to 24 C-atoms, in which one or more —CH₂— groups are optionally substituted by —CH═CH— and/or —O— and/or in which one or more H-atoms are optionally substituted by —OH, and
R³ is H or a linear, branched, or cyclic alkyl group with 1 to 24 C-atoms, in which one or more —CH₂— groups are optionally substituted by —CH═CH— and/or —O— and/or in which one or more H-atoms are optionally substituted by —OH.

4. The pharmaceutical composition according to claim 1, wherein the skin permeation agent is the amide of formula II 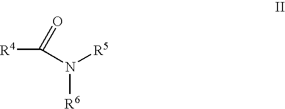
wherein:
R⁴ is a linear, branched, or cyclic alkyl group with 4 to 20 C-atoms, in which one or more —CH₂— groups are optionally substituted by —CH═CH— and/or —O— and/or in which one or more H-atoms are optionally substituted by —OH,
R⁵ is an alkanolyl group with 2 to 8 C-atoms, and
R⁶ is H or an alkanolyl group with 2 to 8 C-atoms.

5. The pharmaceutical composition according to claim 1, wherein the skin permeation enhancing agent is ethanolamine, diethanolamine, triethanolamine, propanolamine, diisopropanolamine, triisopropanolamine, butanolamine, dibutanolamine, or tributanolamine.

6. The pharmaceutical composition according to claim 1, wherein the skin permeation enhancing agent is polyoxyethylene oleylamine, polyoxyethylene stearylamine, polyoxyethylene myristylamine and polyoxyethylene laurylamine, wherein the polyoxyethylene group consists of 5 to 30 ethylene oxide units.

7. The pharmaceutical composition according to claim 1, wherein the skin permeation enhancing agent is coconut fatty acid diethanolamide or lauric fatty acid diethanolamide.

8. The pharmaceutical composition according to claim 1, wherein the amount of meloxicam or a pharmaceutically acceptable salt thereof is from 0.005 weight-% to 10 weight-% of the composition, and the amount of the permeation enhancing agent is from 0.05 weight-% to 20 weight-% of the composition.

9. The pharmaceutical composition according to claim 1, further comprising a supplementary permeation enhancing agent selected from the group consisting of terpenes, terpene alcohols, fatty acids, fatty acid esters, and fatty alcohols.

10. The pharmaceutical composition according to claim 9, wherein the supplementary permeation enhancing agent is 1-menthol, eucalyptus oil, mentha oil, cineole, or limonene.

11. The pharmaceutical composition according to claim 9, wherein the supplementary permeation enhancing agent is capric acid, oleic acid, palmitic acid, lauric acid, myristic acid, stearic acid, or isostearic acid.

12. The pharmaceutical composition according to claim 9, wherein the supplementary permeation enhancing agent is diisopropyl adipate or isopropyl myristate.

13. The pharmaceutical composition according to claim 9, wherein the supplementary permeation enhancing agent is lauryl alcohol, oleyl alcohol, stearyl alcohol, caprylic alcohol, and myristyl alcohol.

14. The pharmaceutical composition according to claim 9, wherein the amount of the supplementary permeation enhancing agent is 0.05 weight-% to 60 weight-% of the composition.

15. The pharmaceutical composition according to claim 1, further comprising at least one adhesive, gelling, and/or thickening agent.

16. The pharmaceutical composition according to claim 1, wherein the adhesive, gelling, and/or thickening agent is selected from the group consisting of gum based adhesives or polymers or copolymers based on acrylates, cellulose ethers, vinyl alcohols, vinyl pyrrolidones, polyoxyethylenes, and/or polyoxypropylenes.

17. The pharmaceutical composition according to claim 15, wherein the amount of the adhesive, gelling, and/or thickening agent is 1.0 weight-% to 99 weight-% of the composition.

18. The pharmaceutical composition according to claim 1, further comprising at least one agent selected from the group consisting of volatile or non-volatile solubilizing agent, pH modifying agent, humectant, moisturizer, preservative, opacifier, fragrances, color additives, and counter-irritants.

19. The pharmaceutical composition according to claim 1, wherein the composition is a gel, a spray, an aerosol, a lotion, a cream, an ointment, or a paste.

20. A transdermal delivery system comprising the pharmaceutical composition according to claim 1 in a matrix system.

21. A transdermal delivery system comprising the pharmaceutical composition according to claim 1 in an adhesive matrix patch.

22. A transdermal delivery system comprising the pharmaceutical composition according to claim 1 in a liquid reservoir system.

23. A transdermal delivery system comprising the pharmaceutical composition according to claim 1 in a cataplasm and/or a liquid reservoir patch.
